(19) United States
(12) Patent Application Publication
(10) Pub. No.: US 2021/0308190 A1
(43) Pub. Date: Oct. 7, 2021

(54) HUMAN UMBILICAL CORD BLOOD MESENCHYMAL STEM CELL TRANSFUSION IMMUNOTHERAPY FOR TREATMENT OF CYTOKINE STORM ASSOCIATED WITH CORONAVIRUS INFECTION

(71) Applicant: MIGUELANGELO J. PEREZ-CRUET, Bloomfield, MI (US)

(72) Inventor: MIGUELANGELO J. PEREZ-CRUET, Bloomfield, MI (US)

(21) Appl. No.: 17/217,065

(22) Filed: Mar. 30, 2021

Related U.S. Application Data
(60) Provisional application No. 63/006,283, filed on Apr. 7, 2020.

(51) Int. Cl. A61K 35/51; A61K 9/00; C12N 5/0775

(52) U.S. Cl. CPC A61K 35/51; C12N 5/0665; A61K 9/0019

(57) ABSTRACT

A method for administering human umbilical cord blood mesenchymal stem cell transfusion immunotherapy to a patient for treatment of coronavirus infection. The method includes harvesting umbilical cord mesenchymal stem cells, culturing the stem cells, collecting and purifying the cultured stem cells, placing the purified stem cells into a transfusion bag, and intravenously transfusing the stem cells from the transfusion bag into the patient having the infection.

### CROSS REFERENCE RELATED TO APPLICATIONS

This application claims the benefit of the filing date of Provisional Application No. 63/006,283, titled, Human Umbilical Cord Blood Mesenchymal Stem Cell Transfusion Immunotherapy for Treatment of Cytokine Storm Associated with Coronavirus Infection, filed on Apr. 7, 2020.

### BACKGROUND

### Field

This disclosure relates generally to a method for treating a virus and, more particularly, to a method for administering human umbilical cord blood mesenchymal stem cell (hUCBMSC) transfusion immunotherapy for treatment of coronavirus infection.

### Discussion of the Related Art

The angiotensin converting enzyme (ACE2) receptor is widely distributed on human cell surfaces, especially the alveolar type II cells (AT2) and capillary endothelium. The AT2 cells also highly express transmembrane serine protease 2 (TMPRSS2), an enzyme. In addition to the lungs, the ACE2 receptor is widely expressed in human tissues, including the heart, liver, kidney and digestive organs. Almost all endothelial cells and smooth muscle cells in organs express ACE2 receptors. Therefore, once a virus enters the blood circulation, it spreads widely by entering targeted cells via the ACE2 receptor and TMPRSS2. However, in the bone marrow, lymph nodes, thymus and the spleen, immune cells, such as T and B-lymphocytes, and macrophages are consistently negative for ACE2. When the COVID-19 virus infects lung tissue it can cause a cytokine storm, resulting in the release of IL-2, IL-6, IL-7, GSCF, IP10, MCP1, MIP1A, and TNF-7, GSCF, IP10, MCP1, MIP1A, and TNFα, followed by pulmonary edema, dysfunction of air exchange, SARS, acute cardiac injury and secondary infection, which may lead to death. Armed with this knowledge, treatments for COVID-19 infections can be devised.

### SUMMARY

The following discussion discloses and describes a method for administering human umbilical cord blood mesenchymal stem cell (hUCBMSC) transfusion immunotherapy to a patient for treatment of coronavirus infection. The method includes harvesting umbilical cord mesenchymal stem cells, culturing the stem cells, collecting and purifying the cultured stem cells, placing the purified stem cells into a transfusion bag, and intravenously transfusing the stem cells from the transfusion bag into the patient having the infection.

Additional features of the disclosure will become apparent from the following description and appended claims, taken in conjunction with the accompanying drawings.

### BRIEF DESCRIPTION OF THE DRAWINGS

FIG. 1 is a flow chart diagram illustrating a method for administering hUCBMSC transfusion immunotherapy for treatment of COVID-19 infection;

FIG. 2 is an illustration of a human umbilical cord being harvested;

FIG. 3 is an illustration of mesenchymal stem cells being collected from the harvested umbilical cord;

FIG. 4 is an illustration of collected umbilical cord mesenchymal stem cells being cultured;

FIG. 5 is an illustration of a tube holding umbilical cord mesenchymal stem cells after they have been removed from the culture dish and centrifuged; and

FIG. 6 is an illustration of a clinical setting showing a patient receiving an hUCBMSC transfusion.

### DETAILED DESCRIPTION OF THE EMBODIMENTS

The following discussion of the embodiments of the disclosure directed to a method for administering human umbilical cord blood mesenchymal stem cell (hUCBMSC) transfusion immunotherapy for treatment of coronavirus infection is merely exemplary in nature, and is in no way intended to limit the disclosure or its applications or uses.

This disclosure proposes that an optimal therapeutic strategy for COVID-19 treatment is intravenous (IV) hUCBMSCs transfusion immunotherapy. This immunotherapeutic strategy has the benefits of eliminating the cytokine storm caused by COVID-19 viral infectivity of the pulmonary system while providing trophic factors critical to multi-organ system recovery. Immunomodulation therapy using mesenchymal stem cells (MSCs) has been suggested to weaken the cytokine storm seen in severe pneumonia caused by influenza, which is similar to the pathophysiological condition causing SARS in critically ill COVID-19 patients. Mesenchymal stem cells (MSCs) have been shown to treat a number of conditions including type 2 diabetes, graft versus host disease, spinal cord injury, and other pathology with good clinical efficacy and safety. A recent study has even shown the clinical efficacy of curing AIDS with a patient showing no HIV markers in the blood after 30 months following blood stem cell treatment. Initial clinical investigations preformed in China using stem cell transfusions have shown extremely promising results in the treatment of COVID-19 infections in critically ill patients.

Currently, most stem cell based clinical trials involve autograph stem cells taken from the patient to treat his or her condition with less than optimal clinical results. Autograph stem cells taken from an adult may not be as effective as allograft hUCBMSCs due to their relative lack of pluripotent potential. Additionally, autograph cells taken from mature individuals can potentially be targeted by pathogens, i.e., the COVID-19 virus, or the immune system due to differentiation of cell surface receptor antigens or major histocompatibility complexes. Initial clinical studies have revealed that allogeneic MSCs transfused into COVID-19 infected patients did not develop the ACE2 receptor targeted by the virus.

The pathogenesis of lung injury in COVID-19 infected patients is the result of a cytokine storm. When the immune system responds to the COVID-19 viral pathogen it can recruit large numbers of inflammatory cells and factors that end up attacking the patient's infected cells along with the healthy cells resulting in a cytokine storm. To illustrate this scientific understanding, a similar phenomenon to cytokine storm has been observed when treating malignant brain tumors using adenoviral vector thymidine kinase gene therapy. Even though only a small percentage of the tumor cells took up the adenoviral vector, all tumor cells were eliminated following treatment with ganciclovir due to an inflammatory effect, which recruited macrophages and lymphocytes to remove non-infected tumor cells. The complete killing of all tumor cells has been achieved due to this bystander effect. A similar situation is occurring in those critically ill COVID-19 infected patients experiencing a cytokine storm resulting in lung injury and the need for ventilator respiratory support. This results in a clinical downward trend to multi-organ failure and death.

The cytokine storm induced by COVID-19 viral-triggered infection results in acute cytokine release of IL-2, IL-6, IL-7, GSCF, IP10, MCP1, MIP1A and TNF. This induces pulmonary edema, dysfunction of air-exchange, SARS, acute cardiac injury, and often-secondary infection, leading to death. Leukemia inhibitory factor (LIF) is known to be indispensable to oppose the cytokine storm in the lungs during viral pneumonia. A recent Lancet publication revealed the death rate of COVID-19 is 10 fold higher than Influenza A. In hospital death is associated with increasing age and significant correlation with IL-6. Though epidemiological data vary from country to country based on mitigation, i.e., social distancing, estimates of the severity of COVID-19 infection indicate 80% asymptomatic to mild disease, 14% severe and 6% critically ill.

The hUCBMSCs have shown very significant immunomodulation and tissue repair effects with low immunogenicity, which makes them an ideal candidate to the allogeneic adoptive transfusion therapy. The immunomodulatory effects of hUCBMSCs is mainly due to the paracrine effects of humoral factors, such as interleukin (IL)-6, IL-8, vascular endothelial growth factor, collagen and elastin, rather than the multi-lineage and regenerative capacities of the stem cells. Stem cell preparations derived from hUCBMSCs, including conditioned media and exosomes, remain a pre-clinical technology despite their great clinical potential. Higher serum concentrations of certain cytokines (IL-1β, IL-6, IL-8, IL-10, IFN-γ) and lower concentrations of other cytokines (IL-17, RANTES, and TNF-β) were associated with cytokine storm development of bronchopulmonary dysplasia death in infants. Cytokine storm seen in infant death caused by bronchopulmonary dysplasia is similar to the clinical and pathophysiologic scenario seen in critically ill COVID-19 patients dying from pulmonary complications. A first-in-human clinical trial of hUCBMSCs treatment for bronchopulmonary dysplasia in infants was performed as a phase I dose-escalation trial. That trial demonstrated the short and long-term safety and feasibility of hUCBMSCs transfusion immunotherapy in treating bronchopulmonary dysplasia. The hUCBMSCs transfusion immunotherapy significantly reduced inflammatory marker expression observed in tracheal aspirates resulting in survival and recovery of infants who would have otherwise died. Thus, there exists human clinical trial safety and efficacy data to proceed with novel hUCBMSCs transfusion immunotherapy for patients experiencing the deleterious effects of coronavirus induced cytokine storm. Therapy using hUCBMSCs was also suggested to be a potential treatment for H5N1 infection induced acute lung injury, which showed a similar inflammatory cytokine profile to that of COVID-19. It has been shown that hUCMSCs can be easily harvested and cultured. It is believed that immunotherapy hUCBMSCs transfusion immunotherapy is safe and effective for critically ill COVID-19 infected patients.

Two particular initial clinical studies from China and not available on pubmed, one a case report and another a 7 patient series, show extremely promising results of transfusion using hUCBMSC or MSC immunotherapy, respectively, and illustrate remarkable repair of injured lung tissue in critically ill COVID-19 patients. These clinical studies showed how the patient's own immune system bolstered by allogeneic pluripotent MSCs could counteract the cytokine storm induced SARS. A case report documented a 65-year old COVID-19 positive critically ill ventilator dependent female patient with elevated liver enzymes was treated with allogeneic hUCBMSCs transfusion. Glucocorticoid and anti-viral therapy failed. Prior to treatment, the immunotherapeutic hUCBMSC transfusion method was discussed and approved by the ethics committee of the hospital and treatment consent forms signed by family members. The allogeneic hUCBMSCs were produced under GMP conditions and administered intravenously at three times (5×10⁷ cells each transfusion, or approximately 750,000 cell/kg in a 70 kg adult) on Feb. 9, 12, and 15, 2020. During the transfusion therapy, thymosin al (a naturally occurring thymic peptide that stimulates the development of immune T cells) and antibiotics were given to boost the immune response and prevent infection, respectively. On Feb. 13, 2020, after just two transfusions of hUCBMSCs, the patient was extubated and started to ambulate. Improvements in blood cell counts were noted after the second transfusion. The counts of CD3+ T cell, CD4+ T cell and CD8+ T cell increased to normal levels. On Feb. 17, 2020 the patient was transferred out of ICU, and most of her vital signs and clinical laboratory indexes recovered to normal levels. The throat swabs and PCR analysis for COVID-19 tests were reported negative on both Feb. 17 and Feb. 19, 2020. The authors suggested that the immune modulating effects of thymosin al alone (from day 7 to day 12) was not significant, but that hUCBMSCs immunotherapy transfusion alone or in combination with thymosin al greatly reduce the inflammatory response caused by the COVID-19 cytokine storm and aided in recovery of patient's antiviral immune system. Laboratory analysis revealed circulating lymphocytic T cell counts returning to normal signaling the end of COVID-19 viral induced inflammatory response within the pulmonary system. The investigators hypothesized that the immunotherapeutic characteristics of hUCBMSCs might repair the injured tissues and neutralize the inflammatory cytokines, such as G-CSF and IL-6, by the expression of their receptors. Further laboratory investigation, isolation and characterization of COVID-19 patient's transfused hUCBMSCs could determine this. No adverse events were observed in this patient receiving hUCBMSCs immunotherapy transfusion. Study investigators concluded that the adoptive transfusion therapy using hUCBMSCs might be an ideal choice or combined with other immune modulating agents to treat critically ill COVID-19 patients and encouraged further investigation.

A patient series in China was conducted enrolling 7 patients in a phase 1 clinical trial to receive MSC transfusion therapy. This clinical trial included 7 patients with COVID-19 induced pneumonia: 1 critically ill, 4 severe, and 2 non-severe. All patients had high fevers, shortness of breath and poor oxygen saturation. A single clinical grade MSC transfusion dosage 1×10⁶ MSC/kg weight was given. Within 2 days all patients displayed clinical improvement with a patient with severe symptoms able to be discharged on day 10 post-transfusion. Laboratory analysis revealed peripheral lymphocytes increased with a shift towards the normal phenotype for both CD4+ T cells and dendritic cells; and inflammatory cytokines significantly decreased while IL-10 increased. This clinical pilot study also investigated the fate of the MSC transfused cells. The transfused MSCs did not acquire the ACE2 receptor, but did show beneficial high levels of anti-inflammatory and trophic factor activity including TGF, HGF, LIF, VEGF, EGF, BDNF and NGF, demonstrating that the immunomodulation properties of the MSC are long-term and maintained by cytokine production. Leukemia inhibitory factor (LIF) released by MSCs is critical in controlling and stopping the cytokine storm produced by COVID-19 pulmonary infections. At 2 to 4 days after MSCs transfusion, all symptoms disappeared in all the patients, oxygen saturations rose to 95% at rest without or with oxygen uptake (5 liters per minute). In addition, no acute infusion-related or allergic reactions were observed within two hours after transplantation. Similarly, no delayed hypersensitivity or secondary infections were detected after treatment. Of note, in the COVID-19 critically ill patient with a history of stage 3 hypertension, pre-transfusion analysis indicated liver and myocardium injury with elevated 57 U/L aspartic aminotransferase, 513 U/L creatine kinase activity and 138 ng/ml myoglobin levels, respectively. However, 2 to 4 days post-transfusion the levels of these functional biomarkers decreased to normal reference values: 19 U/L, 40 U/L, and 43 ng/ml, respectively, indicating the multi-organ restorative efficacy of MSCs transfusion. Chest CT radiographic analysis showed that the ground-glass opacity and pneumonia infiltrates had largely resolved by the 9th day post transfusion thus preventing long-term permanent pulmonary fibrosis as seen similarly in infants suffering from bronchopulmonary dysplasia treated with hUCBMSCs transfusion. The pre-transfusion percentages of T and NK cells were markedly increased due to cytokine storm. However, 6 days post MSC transfusion, the concentrations of these cells nearly disappeared and other immune cell subpopulations were almost restored to the normal levels, especially the CD14+CD11c+CD11cmid regulatory dendritic cell population. Furthermore, the investigation revealed that transfused MSCs were ACE2 or TMPRSS2 negative, indicating that MSCs were immune to COVID-19 infection. Moreover, anti-inflammatory and trophic factors like TGF-β, HGF, LIF, GAL, NOA1, FGF, VEGF, EGF, BDNF, and NGF were highly expressed in MSCs, demonstrating the immunomodulatory function of MSCs and potential clinical efficacy. Ultimately the mechanism of action was felt due to a unique immunosuppressive capacity of MSCs to reduce serum levels of pro-inflammatory cytokines and chemokines, which resulted in less mononuclear/macrophages migration to fragile lung tissue, while inducing regulatory trophic dendritic cells to benefit multi-organ healing.

The above mentioned clinical and laboratory results revealed a novel hUCBMSCs transfusion immunomodulation therapy for treating coronavirus, like COVID-19, critically ill patients and could well stem the death rate and morbidity for this disorder.

Patients to receive transfusion of medical grade hUCBMSCs produced using good manufacturing product facility guidelines after FDA approval to conduct the trial. The hUCBMSCs immunotherapy transfusion will be initiated when the patient's symptoms and/or signs are getting progressively worse and there appears to be no other viable treatment option.

The patient will be monitored in an intensive care unit (ICU) setting. Clinical assessment will include pre and post-transfusion variables including but not limited to vitals, heart rate and heart pattern, temperature, changes in behavior, i.e., irritability, changes in respiratory rate, oxygenation, perfusion of extremities seen with finger oxygenation probe. The hUCBMSCs in a concentration of 1×10⁶ cells per kilogram patient weight will be suspended in 100 ml of sterile normal saline. An IV line will be placed in the patient's arm. The transfusion rate will be set at 2 ml per minute or 120 ml per hour. A slower rate of 60 ml per hour can also be used to prevent further pulmonary edema. Thus, transfusion could take approximately 1-2 hours. Patients will be monitored in an ICU setting and vital signs and clinical parameters recorded hourly. Discontinuation of transfusion will be performed if the patient develops an allergic reaction or is unable to maintain adequate oxygen saturation despite ventilation support or in the event CPR is required. Additional information included primary safety data such as transfusion related allergic reaction, secondary infection and adverse events will be documented. The primary efficacy data such as levels of the cytokines variation, C-reactive plasma proteins and oxygen saturation levels will be documented. Also, the secondary efficacy outcomes included total lymphocyte count and subpopulations, chest CT, respiratory rate, and the patient symptoms (especially fever and shortness of breath) will be recorded. In addition, ongoing therapeutic measures, i.e., antiviral medicine and respiratory support, and daily patient outcomes will be examined. Routine laboratory analysis including CBC, electrolytes, liver function test will be performed daily while hospitalized and in accordance with good medical practice.

Pre and post transfusion clinical, laboratory and radiographic evaluation for enrolled study patients will be conducted according to the Southeast Michigan COVID-19 Consortium Case Report Form. In addition, a thorough analysis of the study patient's pre- and post-operative immune cellular response will be conducted to collect data on T cell and NK cell response.

RT-PCR analysis of HCoV-19 nucleic acid will be performed before and after hUCBMSC transfusion. Repeat analysis for COVID-19 positivity in transfused patients will be conducted at 3, 6, 10, 14 and 20 days post transfusion. COVID-19 antibody production analysis will also be conducted at 3, 6, 10, 14 and 20 days post transfusion. This time line is based on data provided by the pilot study preformed. In that particular study, it was determined that at 6 days post-transfusion patients remained COVID-19 positive and turned COVID-19 negative at day 13 post-transfusion. This time line in documenting post-transfusion negativity and antibody production can help guide a better understanding of patient's immune response to COVID-19 infection treated with hUCBMCS transfusion.

Autoimmune cell count analysis of peripheral lymphocytes, T and NK cells, as well as CD14+CD11c+CD11bmid regulatory DC cells will be analyzed pre and post-hUCBMSC transfusion at 3, 6, 10, 14, and 20 days. It was determined that peripheral lymphocytes were increased and the over activated cytokine-secreting T and NK immune cells disappeared in 3-6 days post-transfusion while a group of CD14+CD11c+CD11bmid regulatory DC cell population dramatically increased.

The transfused population of hUCBMSCs will be analyzed for acquisition of ACE2 receptors via polymerize chain reaction (PCR) determination. Levels of TNF-α and IL-10 will also be investigated. Clinical series determined that TNF-α significantly decreased while IL-10 increased in MSC treatment group compared to placebo control MSC group. Furthermore, study revealed that gene expression profile showed MSCs were ACE2 and TMPRSS2 receptor negative and thus immune to COVID-19 infection.

The patients will be assessed daily while hospitalized. Clinical follow-up in the event of discharge from the hospital will be conducted at 2 week, 2, 4, 6, 12 and 24 months post hospital discharge and when deemed necessary by the clinician. Routine clinical, laboratory, and radiologic investigation will be done after discharge from the hospital and patient outcomes recorded by a certified group of doctors. The detailed record included primary safety data (transfusion and allergic reactions, secondary infection and adverse events) and the primary efficacy data (the level of the cytokines variation, the level of C-reactive protein in plasma and the oxygen saturation) will be recorded. The secondary efficacy outcomes mainly included the total lymphocyte count and subpopulations, the chest CT, the respiratory rate and the patient symptoms (especially the fever and shortness of breath). In addition, the therapeutic measures, i.e., antiviral medicine and respiratory support, and outcomes will also be documented. Bi-daily lab analysis will be conducted doing a full panel including CBC, electrolytes panel.

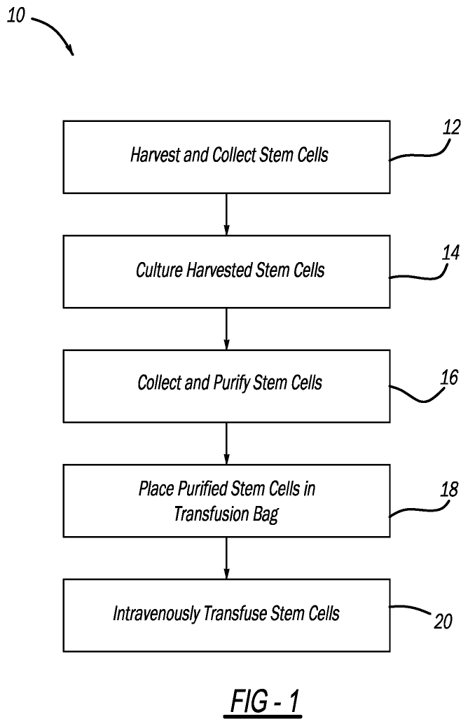

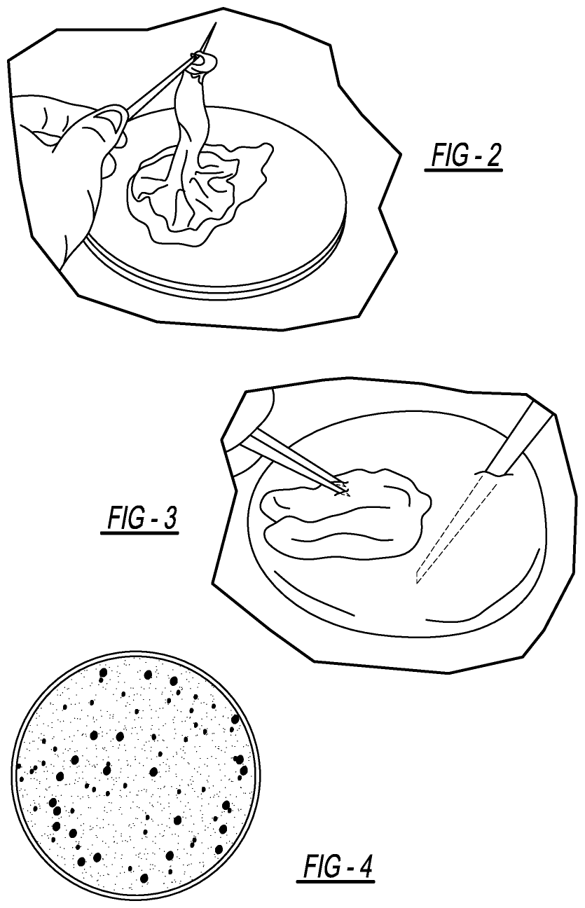

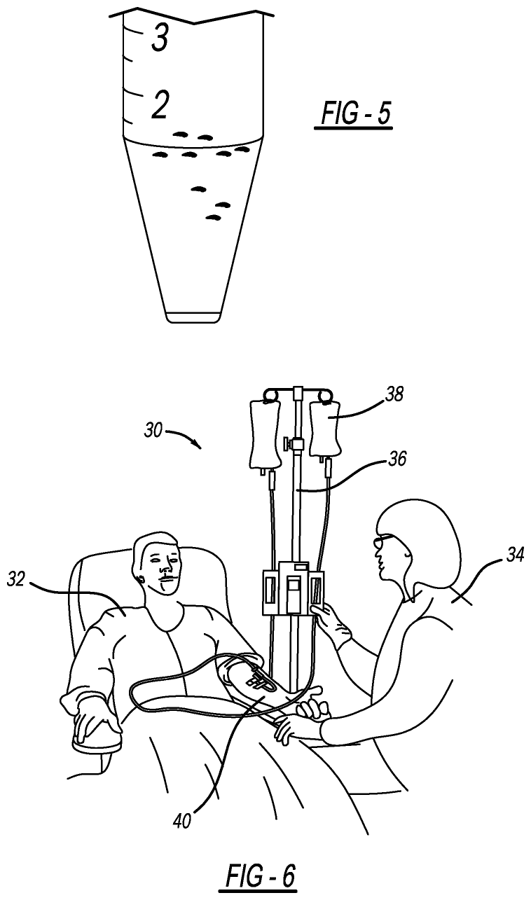

FIG. 1 is a flow chart diagram 10 showing a method for administering hUCBMSC transfusion immunotherapy for treatment of COVID-19 infection consistent with the discussion above. The umbilical cord mesenchymal stem cells are harvested and collected at box 12. FIG. 2 is an illustration of a human umbilical cord being harvested and FIG. 3 is an illustration of mesenchymal stem cells being collected from the harvested umbilical cord. The collected umbilical cord mesenchymal stem cells are then cultured or grown at box 14. FIG. 4 is an illustration of umbilical cord mesenchymal stem cells being cultured in a culture dish. The cultured umbilical cord mesenchymal stem cells are then collected from the culture dish and purified at box 16. FIG. 5 is an illustration of a tube holding a pellet of umbilical cord mesenchymal stem cells after they have been removed from the culture dish and centrifuged. The purified stem cells are then placed in a transfusion bag at box 18, and intravenously transfused from the transfusion bag into a patient at box 20.

FIG. 6 is an illustration of a clinical setting 30 showing a patient 32 receiving an hUCBMSC transfusion. A technician 34 is shown monitoring an IV stand 36 holding a transfusion bag 38 of prepared hUCBMSCs that are administered intravenously into an arm 40 of the patient 32.

The foregoing discussion discloses and describes merely exemplary embodiments of the present disclosure. One skilled in the art will readily recognize from such discussion and from the accompanying drawings and claims that various changes, modifications and variations can be made therein without departing from the spirit and scope of the disclosure as defined in the following claims.

What is claimed is:

1. A method for treating a patient having a coronavirus infection, said method comprising:
harvesting and collecting umbilical cord mesenchymal stem cells;
culturing the stem cells;
collecting and purifying the cultured stem cells;
placing the purified stem cells into a transfusion bag; and
intravenously transfusing the stem cells from the transfusion bag into the patient.

2. The method according to claim 1 wherein placing the purified stem cells includes placing a concentration of 1×10⁶ cells per kilogram patient weight suspended in 100 ml of sterile normal saline.

3. The method according to claim 1 wherein intravenously transfusing the stem cells includes using a transfusion rate of 120 ml per hour.

4. The method according to claim 1 wherein intravenously transfusing the stem cells includes using a transfusion rate of 60 ml per hour.

5. The method according to claim 1 further comprising documenting primary efficacy data including levels of cytokines variation, C-reactive plasma proteins and oxygen saturation levels during the transfusion.

6. The method according to claim 1 further comprising recording secondary efficacy outcomes including total lymphocyte count and subpopulations, chest CT, respiratory rate and patient symptoms.

7. The method according to claim 1 further comprising examining ongoing therapeutic measures including antiviral medicine, respiratory support, and daily patient outcome.

8. The method according to claim 1 wherein the coronavirus infection is COVID-19.

9. The method according to claim 1 wherein the umbilical cord mesenchymal stem cells are human umbilical cord mesenchymal stem cells.

10. A method for administering human umbilical cord blood mesenchymal stem cell transfusion immunotherapy to a patient for treatment of coronavirus infection.

11. The method according to claim 10 wherein the coronavirus infection is COVID-19.

12. A system for treating a patient having a coronavirus infection, said system comprising:
means for harvesting and collecting umbilical cord mesenchymal stem cells;
means for culturing the stem cells;
means for collecting and purifying the cultured stem cells;
means for placing the purified stem cells into a transfusion bag; and
means intravenously transfusing the stem cells from the transfusion bag into the patient.

13. The system according to claim 12 wherein the means for placing the purified stem cells places a concentration of 1×10⁶ cells per kilogram patient weight suspended in 100 ml of sterile normal saline.

14. The system according to claim 12 wherein the means for intravenously transfusing the stem cells uses a transfusion rate of 120 ml per hour.

15. The system according to claim 12 wherein the means for intravenously transfusing the stem cells uses a transfusion rate of 60 ml per hour.

16. The system according to claim 12 further comprising means for documenting primary efficacy data including levels of cytokines variation, C-reactive plasma proteins and oxygen saturation levels during the transfusion.

17. The system according to claim 12 further comprising means for recording secondary efficacy outcomes including total lymphocyte count and subpopulations, chest CT, respiratory rate and patient symptoms.

18. The system according to claim 12 further comprising means for examining ongoing therapeutic measures including antiviral medicine, respiratory support, and daily patient outcome.

19. The system according to claim 12 wherein the coronavirus infection is COVID-19.

20. The system according to claim 12 wherein the umbilical cord mesenchymal stem cells are human umbilical cord mesenchymal stem cells.
